(19) United States
(12) Patent Application Publication
(10) Pub. No.: US 2024/0279667 A1
(43) Pub. Date: Aug. 22, 2024

(54) COMPOSITIONS AND METHODS OF TREATING DISEASES ASSOCIATED WITH BILE ACID TRANSPORTER

(71) Applicant: HemoShear Therapeutics, Inc., Charlottesville, VA (US)

(72) Inventors: Ryan FEAVER, Charlottesville, VA (US); Brian JOHNS, Charlottesville, VA (US); Brian WAMHOFF, Charlottesville, VA (US)

(21) Appl. No.: 18/634,110

(22) Filed: Apr. 12, 2024

Related U.S. Application Data
(63) Continuation of application No. PCT/US2022/078103, filed on Oct. 14, 2022.
(60) Provisional application No. 63/255,701, filed on Oct. 14, 2021.

(51) Int. Cl. C12N 15/113

(52) U.S. Cl. CPC C12N 15/1138; C12N 2310/14; C12N 2310/351

(57) ABSTRACT

The present disclosure provides nucleic acid based therapeutics, such as small interfering ribonucleic acids (siRNAs), which repress expression of human SLC10A1 mRNA transcripts for treating NTCP associated diseases and disorders. Also provided herein are methods of treating cholestatic disorders, HDV, HBV, NAFLD, and NASH with the siRNAs, conjugates and compositions provided herein.

### CROSS REFERENCE TO RELATED APPLICATIONS

This application claims priority to U.S. Provisional Application No. 63/255,701, filed on Oct. 14, 2021; the contents of which are incorporated herein by reference in their entirety.

### DESCRIPTION OF THE TEXT FILE SUBMITTED ELECTRONICALLY

The application is being filed with an electronically submitted Sequence Listing file in XML format; the file is named as HEM-001WO1_SL.XML, created on Oct. 13, 2022, which has a size of 12,689 kilobytes; the contents of which are incorporated herein by reference in their entirety.

### BACKGROUND

Cholestatic disorders are associated with high rates of morbidity and mortality, and are the leading cause for pediatric liver transplant. Cholestatic disorders include progressive intrahepatic familial cholestasis (PFIC), Alagille syndrome (ALGS), biliary atresia (BA), primary biliary cholangitis (PBC), primary sclerosing cholangitis (PSC), intrahepatic cholestasis of pregnancy (ICP), ductal plate abnormalities, Caroli syndrome, congenital hepatic fibrosis, and bile acid synthesis defects. Treatment of these conditions typically involves supportive care for complications from these disorders, including treatment for malnutrition, pruritis, and hypertension. There are limited effective interventions to prevent progressive liver damage in these diseases.

Hepatitis D is a liver disease resulting from co-infection or superinfection with the hepatitis D virus (HDV) with hepatitis B virus (HBV). The disease pathology resulting from a HDV and HBV infection can be extremely serious leading to severe complications and a greater likelihood rapid progression to cirrhosis and liver cancer. There are limited effective interventions available for the treatment of these infections resulting in significant unmet medical need.

Sodium taurocholate co-transporting polypeptide (NTCP) is a sodium-dependent uptake transporter residing in the basolateral membrane of hepatocytes and is involved in the hepatic uptake of bile acid from blood. In addition to its transport function, NTCP is also an important entry receptor for hepatitis virus B (HBV) and HDV. The roles of NTCP make it a potential target for treating a cholestatic disorder, Hepatitis B and/or Hepatitis D in a patient. The present invention discloses that silencing and/or downregulation of expression of the gene encoding NTCP, SLC10A1 in the liver can reduce and/or inhibit NTCP mediated activities, thereby treating a liver disease, e.g., a cholestatic disorder, hepatitis B, hepatitis D, NAFLD and/or NASH.

### SUMMARY

The present invention provides among other things, nucleic acid based therapeutics for effectively targeting NTCP (Na⁺-taurocholate cotransporting polypeptide) and for treating diseases and/or disorders that are associated with NTCP. In particular, the present disclosure provides siRNAs to silence and/or downregulate expression of SLC10A1, the gene encoding NTCP protein.

Provided herein are compounds which solve the need in the art for treatment of cholestatic disorders, hepatitis B and hepatitis D, and/or NAFLD and NASH. Also provided herein are methods for degrading mRNA transcripts of the human SLC10A1 gene and methods for treating cholestatic disorders, HDV and HBV infections, NAFLD and NASH in a patient in need.

In one aspect of the present invention, provided herein is a compound comprising a small interfering ribonucleic acid sequence (siRNA) which targets a human SLC10A1 mRNA transcript that encodes NTCP. The siRNA can repress translation of the human SLC10A1 gene that encodes NTCP, thereby reducing and/or preventing NTCP mediated activities.

In some embodiments, the siRNA comprises a nucleic acid sequence at least 70%, at least 75%, at least 80%, at least 85%, at least 90%, at least 95%, at least 96%, at least 97%, at least 98%, at least 99%, or 100% complementary to a portion of the nucleic acid sequence of the human SLC10A1 mRNA transcript (i.e., a targeting sequence). The targeting sequence within the human SLC10A1 mRNA transcript may locate in the 3′ end untranslated region (3′UTR), the coding region, and/or the 5′ end UTR region of the human SLC10A1 mRNA. As non-limiting examples, the siRNA is complementary to a portion of the nucleic acid sequence encoding the amino acid sequence of SEQ ID NO: 11. In some embodiments, the siRNA comprises a nucleic acid sequence complementary to a portion of the nucleic acid sequence of SEQ ID NO: 1, or to a portion of the nucleic acid sequence of SEQ ID NO: 2.

In some embodiments, the siRNA comprises about 12-30 nucleotides, or about 15-25 nucleotides, or about 17-25 nucleotides. As non-limiting examples, the siRNA comprises 17, 18, 19, 20, 21, 22, 23, 24, 25, 26, 27, 28, 29 or 30 nucleotides. In one embodiment, the siRNA comprises 19 nucleotides. In one embodiment, the siRNA comprises 21 nucleotides. In another embodiment, the siRNA comprises 23 nucleotides. In some embodiments, the siRNA comprises a sense strand and an antisense strand, wherein the sense and antisense strands form a duplex. As non-limiting examples, the sense strand of the siRNA comprises 19 nucleotides and the antisense strand of the siRNA comprises 21 nucleotides, In another example, the sense strand of the siRNA comprises 21 nucleotides and the antisense strand of the siRNA comprises 23 nucleotides. In some embodiments, the sense and antisense strands of the siRNA are connected as a single strand through a hairpin loop.

In some embodiments, the siRNA targeting a human SLC10A1 mRNA transcript comprises at least one chemical modification, including but not limited to sugar modification, backbone modification and/or nucleobase modification.

In some embodiments, the siRNA targeting a human SLC10A1 mRNA transcript is conjugated to one or more of N-acetyl-D-galactose (GalNAC), cholesterol, lipid, lipophilic molecule, polymer, peptide, ligand, or antibody.

In some embodiments, the siRNA targeting a human SLC10A1 mRNA transcript specifically represses translation of SLC10A1 mRNA in the liver, for example in the liver cells including but not limited to hepatocytes, hepatic stellate cells, Kupffer cells, and liver sinusoidal endothelial cells. In some embodiments, the expression of the human SLC10A1 mRNA transcript is reduced about 20%, 30%, 40%, 45%, 50%, 55%, 60%, 65%, 70%, 75%, 80%, 85%, 90%, 95%, 96%, 97%, 98% or 99%, as compared to normal expression level (e.g., without the treatment of the siRNA described herein).

In some embodiments, the siRNA targeting a human SLC10A1 mRNA transcript can degrade the human SLC10A1 mRNA transcript in a cell (e.g., in a liver cell), wherein the human SLC10A1 mRNA transcript is degraded for at least 2 days, 5 days, 1 week, 2 weeks, or longer. In some embodiments, about 20%, 30%, 40%, 45%, 50%, 55%, 60%, 65%, 70%, 75%, 80%, 85%, 90%, 95%, 96%, 97%, 98% or 99% of the human SLC10A1 mRNA transcript is degraded by the siRNA.

In some embodiments, the siRNA is conjugated to one or more N-acetyl-D-galactose.

In some embodiments, the siRNA has the sequence of any one of SEQ ID NOS: 3-6. In some embodiments, the siRNA targets the 3′ untranslated region of human SLC10A1. In some embodiments, the siRNA targets the coding region of human SLC10A1.

In some embodiments, the siRNA specifically targets human SLC10A1 mRNA transcripts of the liver. In some embodiments, the human SLC10A1 mRNA transcripts are located in a cell selected from the group consisting of hepatocytes, hepatic stellate cells, Kupffer cells, and liver sinusoidal endothelial cells.

In other embodiments, provided herein also include other nucleic acid molecules that can silence and/or downregulate expression of a human SLC10A1 mRNA transcript including but not limited to, dsRNA, shRNA, miRNA, anti-sense oligonucleotide (ASO) and aptamer.

Provided herein also includes a pharmaceutical composition comprising a siRNA and/or other nucleic acid molecules as described herein. The pharmaceutical composition further comprises at least a pharmaceutically acceptable carrier.

In some embodiments, provided herein is a method for degrading a human SLC10A1 mRNA transcript comprising administering a siRNA, a compound, or a composition described herein to a cell. In some embodiments, at least 50% of human SLC10A1 mRNA transcripts are degraded for at least 1 week. In some embodiments, at least 90% of human SLC10A1 mRNA transcripts are degraded for at least 1 week. In some embodiments, at least 95% of human SLC10A1 mRNA transcripts are degraded for at least 1 week. In some embodiments, at least 98% of human SLC10A1 mRNA transcripts are degraded for at least 1 week. In some embodiments, at least 50% of human SLC10A1 mRNA transcripts are degraded for at least 2 weeks. In some embodiments, at least 90% of human SLC10A1 mRNA transcripts are degraded for at least 2 weeks. In some embodiments, at least 95% of human SLC10A1 mRNA transcripts are degraded for at least 2 weeks. In some embodiments, at least 98% of human SLC10A1 mRNA transcripts are degraded for at least 2 weeks.

In another aspect of the present invention, provided herein includes a method for treating a disease that is associated with NTCP in a patient in need; the method comprises administering to the patient a composition comprising a nucleic acid molecule that inhibits (or reduces) expression of a SLC10A1 mRNA transcript that encodes NTCP. The nucleic acid molecule can be a siRNA, a shRNA, a dsRNA, a miRNA, an anti-sense oligonucleotide, or an aptamer. The disease that is associated with NTCP includes but is not limited to a cholestatic disorder, hepatitis B, hepatitis D, NAFLD and NASH. As non-limiting example, the compound to reduce the expression of the SLC10A1 transcript is a siRNA.

In some embodiments, the disease is a cholestatic disorder. In some embodiments, the cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP). In some embodiments, the treatment reduces and/or prevents NTCP mediated bile acid uptake. In some embodiments, after treating the patient exhibits reduced intrahepatic accumulation of bile acids. In some embodiments, after treating the patient experiences an improvement in at least one symptom of a cholestatic disorder. In some embodiments, the symptom is selected from the group consisting of pruritis, mitochondrial damage and inflammation in the liver, and hepatic injury.

In some embodiments, the disease is hepatitis D. In some embodiments, the disease is hepatitis B. In some embodiments, the treatment reduces and/or prevents the NTCP mediated Hepatitis B virus (HBV) interaction and/or Hepatitis D virus (HDV) interaction.

In some embodiments, the disease is NAFLD or NASH. In some embodiments, after treating, the patient experiences an improvement in at least one symptom of NAFLD or NASH, selected from the group consisting of fatty acid metabolism, inflammation and fibrosis. In some embodiments, the method for treating hepatitis B and/or hepatitis D further comprises an anti-viral therapy, or an immunomodulatory therapy, or the combination thereof.

In some embodiments, provided herein is a lipid nanoparticle comprising a compound described herein. In some embodiments, provided herein is a composition containing a compound and/or lipid nanoparticle as described herein. In some embodiments, provided herein is a method of treating a cholestatic disorder in a patient in need thereof comprising administering a nanoparticle and/or composition described herein. In some embodiments, the cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP). In some embodiments, after treating the patient exhibits reduced intrahepatic accumulation of bile acids. In some embodiments, after treating the patient experiences an improvement in at least one symptom of a cholestatic disorder. In some embodiments, the symptom is selected from the group consisting of pruritis, mitochondrial damage and inflammation in the liver, and hepatic injury. Provided herein is a method of treating hepatitis D comprising administering a composition or nanoparticle described herein. Provided herein is a method of treating hepatitis B comprising administering a composition or nanoparticle described herein. Provided herein is a method of treating NAFLD or NASH comprising administering a composition or nanoparticle described herein.

In accordance with the present invention, the nucleic acid molecules, siRNAs, compounds and compositions described herein may be administered subcutaneously, intramuscularly or intravenously.

In yet another aspect of the present invention, provided herein includes a method for blocking Na⁺-taurocholate cotransporting polypeptide (NTCP) mediated activities in the liver of a subject in need comprising administering to the subject with a composition comprising a nucleic acid molecule that targets a human SLC10A1 mRNA transcript directly in the case of ASO therapeutic or through the RNA-induced silencing complex (RISC) in the case of an siRNA therapeutic in the liver, wherein the nucleic acid molecule inhibits expression of the human SLC10A1 mRNA transcript in the liver. The NTCP mediated activities include bile acid uptake in the liver, and HBV and/or HDV interaction.

### BRIEF DESCRIPTION OF THE DRAWINGS

FIGS. 1A-1B shows transport of taurocholic acid (TCA) into control HUH7 cells that do not express SLC10A1 (labeled “Control HUH7 cells”, FIG. 1A) and HUH7 cells overexpressing SLC10A1 (labeled “NTCP Overexpressing HUH7 Cells”, FIG. 1B) after exposure to TCA at concentrations ranging from 0 to 300 μM.

FIG. 2 shows, genes regulated by intracellular bile acid levels via FXR as a surrogate for bile acid uptake, the expression of FGF19 and BSEP gene expression after exposure of primary human hepatocytes to 0 μM, 30 μM, 100 μM, or 300 μM TCA.

FIG. 3A shows expression of SLC10A1 over time in primary human hepatocytes treated with a single treatment of siRNA #4 (an siRNA having the sequence of SEQ ID NO: 6); untreated cells; and cells treated with a control siRNA that does not bind to the SLC10A1 (labeled for non-targeting control, “NTC #1”).

FIG. 3B shows expression of bile acid surrogates FGF19 and BSEP genes in primary human hepatocytes that are treated with a single treatment of siRNA #4 (labeled “NTCP”) or with a control siRNA that does not bind to the SLC10A1 (labeled NTC #1).

FIG. 4A shows expression of SLC10A1 in primary human hepatocytes treated with a single dose of siRNAs #1-4. Cells treated with a control non-targeting siRNA (siRNA NTC) do not have reduced expression of SLC10A1.

FIG. 4B shows reduction in bile acid uptake in primary human hepatocytes treated with a single dose of siRNAs #1-4. Cells untreated or treated with a control non-targeting siRNA (siRNA NTC) were measured as control.

FIG. 5A shows reduction in bile acid uptake in primary human hepatocytes treated with a single treatment of siRNA #4 at different concentrations (0-4 μM).

FIG. 5B shows that correlation of gene expression repression and reduction of bile acid uptake for two exemplary siRNAs: #1 (SEQ ID NO: 3) and #4 (SEQ ID NO: 6)

### DETAILED DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein. Unless otherwise expressly stated, it is in no way intended that any method or aspect set forth herein be construed as requiring that its steps be performed in a specific order. As used herein, the singular forms “a,” “an” and “the” include plural referents unless the context clearly dictates otherwise. As used herein, the term “about” means that the recited numerical value is approximate and small variations would not significantly affect the practice of the disclosed embodiments.

### I. Definitions

Complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. “100% complementarity” or “100% complementary to” refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity.

Coding sequence: As used herein, the term “coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

3′-untranslated region (3′-UTR): A 3′-UTR is typically the part of an mRNA which is located between the protein coding region (i.e. the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3′-end, such as polyadenylation of the 3′-end of the pre-mature mRNA and optional endo- or exonuclease cleavages etc. In the context of the present invention, a 3′-UTR corresponds to the sequence of a mature mRNA which is located 3′ to the stop codon of the protein coding region, preferably immediately 3′ to the stop codon of the protein coding region, and which extends to the 5′-side of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, such as “a 3′-UTR of an albumin gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present invention, a 5′UTR corresponds to the sequence of a mature mRNA which is located between the 5′-CAP and the start codon. Preferably, the 5′-UTR corresponds to the sequence which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a TOP gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

Effective amount: As used herein the term “effective amount” refers to the amount of siRNA or a pharmaceutical composition comprising an siRNA determined to produce a therapeutic response in a mammal. Such therapeutically effective amounts are readily ascertained by one of ordinary skill in the art and using methods as described herein.

Expression: As used herein, the term “expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

mRNA: As used herein, the term “mRNA” refers to a nucleic acid transcribed from a gene from which a polypeptide is translated, and may include non-translated regions such as a 5′UTR and/or a 3′UTR. It will be understood that an siRNA of the invention may comprise a nucleotide sequence that is complementary to any sequence of an mRNA molecule, including translated regions, the 5′UTR, the 3UTR, and sequences that include both a translated region and a portion of either 5′UTR or 3′UTR. An siRNA of the invention may comprise a nucleotide sequence that is complementary to a region of an mRNA molecule spanning the start codon or the stop codon. The term “mRNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

Nucleotide: As used herein, the term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but riot limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Nucleic acid: As used herein, the term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991); Ohtsuka et al., (1985); Rossolini et al., (1994)). In the present disclosure, the terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

Reduce: As used herein, the terms “reduce” or “reducing” or “reduction” refer to silencing, eliminating, knock-down, knock-out, and/or decreasing expression of a target gene. The term “reduced” is used herein to indicate that the target gene expression is lowered by 1-100%. For example, the expression may be reduced by 10, 20, 30, 40, 50, 60, 70, 80, 90, 95, or even 99%. Knock-down of gene expression can be directed by the use of siRNAs or other interfering nucleic acids.

Silence: As used herein, the terms “silence” or “silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a defined, though partially characterized pathway whereby small interfering RNA (siRNA) act in concert with host proteins (e.g., the RNA induced silencing complex, RISC) to degrade messenger RNA (mRNA) in a sequence-dependent fashion. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

The phrase “repressing expression of an SLC10A1 mRNA” as used herein, means administering or expressing an amount of interfering RNA (e.g., an siRNA, a dsRNA, a miRNA, an ASO or an aptamer) to reduce translation of the target SLC10A1 mRNA into protein, either through mRNA cleavage or through direct inhibition of translation. The terms “repressing,” “inhibiting,” “silencing,” and “attenuating” as used herein refer to a measurable reduction in expression of a target mRNA or the corresponding protein as compared with the expression of the target mRNA or the corresponding protein in the absence of an interfering RNA of the invention. The reduction in expression of the target mRNA or the corresponding protein is commonly referred to as “knock-down” and is reported relative to levels present in non-transfected cells or in cells that have been transfected with a control RNA (e.g., a non-targeting control siRNA). Knock-down of expression of an amount including and between 50% and 100% is contemplated by embodiments herein. However, it is not necessary that such knock-down levels be achieved for purposes of the present invention. Knock-down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock-down include RNA solution hybridization, nuclease protection, northern hybridization, gene expression monitoring with a microarray, antibody binding, radioimmunoassay, and fluorescence activated cell analysis.

siRNA: As used herein, the terms “small interfering” or “short interfering RNA” or “siRNA” is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

Subject: As used herein, the terms “subject” and “patient” are used interchangeably. A “subject” or “patient” can be a human or non-human animal.

Target gene: As used herein, “target gene” refers to a nucleic acid sequence in a cell, wherein the expression of the sequence may be specifically and effectively modulated using siRNAs and methods described herein. A “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

Treat: The terms “treatment,” “treating,” “treat” and the like, when used in the context of a disease, injury or disorder, are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The term “percent identity” in the context of two or more nucleic acid or polypeptide sequences, refers to two or more sequences or subsequences that have a specified percentage of nucleotides or amino acid residues that are the same, when compared. Percentage identity can be calculated using the tools CLUSTALW2 or Basic Local Alignment Search Tool (BLAST), which are available online. The following default parameters may be used for CLUSTALW2 Pairwise alignment: Protein Weight Matrix=Gonnet; Gap Open=10; Gap Extension=0.1.

### II. Target Gene SLC10A1

Solute carrier protein (sodium/bile acid cotransporter family, member 1) (NTCP) is a carrier protein in the basolateral membrane of the hepatocyte to uptake bile acids from plasma, playing a crucial role in the enterohepatic circulation of bile acids (Hagenbuch and Dawson, 2004, Pflugers Arch. 447, 566-570). Bile acids are the catabolic product of cholesterol metabolism, hence this protein is important for cholesterol homeostasis. In addition to their role in metabolism, bile acids also act as transcriptional regulators via certain nuclear receptors (NRs). For example, bile acids are strong activators (ligands) for FXR signaling pathway, which plays an important role in balancing bile acids in the liver.

Increased intracellular accumulation of bile acids ultimately results in bile acid-induced hepatocellular damage and apoptosis. NTCP is a key player in this coordinated response designed to help shield the hepatocyte from bile acid damage. The key role of NTCP in cholestasis makes it a superior target for treating a cholestatic disorder.

NTCP is also a functional receptor for hepatitis B virus and hepatitis D virus. It has been shown that preventing interaction between this cell surface receptor and HBV or HDV viral particle can inhibit HBV and HDV infection. NTCP is a potential target for hepatitis B and hepatitis D treatment.

NTCP in humans is encoded by the SLC10A1 (solute carrier family 10 member 1) gene, which is mainly expressed in the liver.

The human NTCP protein (GenBank Reference No: NP_003040.1) comprises an amino acid sequence of SEQ ID NO. 11:

(SEQ ID NO: 11)
MEAHNASAPFNFTLPPNFGKRPTDLALSVILVFMLFFIMLSLGCTMEFS
KIKAHLWKPKGLAIALVAQYGIMPLTAFVLGKVFRLKNIEALAILVCGC
SPGGNLSNVFSLAMKGDMNLSIVMTTCSTFCALGMMPLLLYIYSRGIYD
GDLKDKVPYKGIVISLVLVLIPCTIGIVLKSKRPQYMRYVIKGGMIIIL
LCSVAVTVLSAINVGKSIMFAMTPLLIATSSLMPFIGFLLGYVLSALFC
LNGRCRRTVSMETGCQNVQLCSTILNVAFPPEVIGPLFFFPLLYMIFQL
GEGLLLIAIFWCYEKFKTPKDKTKMIYTAATTEETIPGALGNGTYKGED
CSPCTA

### III. Compounds Comprising siRNAs that Target Human SLC10A1

The present invention provides nucleic acid molecules that can repress expression of a human SLC10A1 mRNA transcript to attenuate NTCP mediated activities for treating diseases that are associated with NTCP. The nucleic acid molecules can degrade the human SLC10A mRNA transcript and/or inhibit its translation. Collectively, the nucleic acid molecules (e.g., DNA, RNA, and DNA or RNA like molecules) of the present disclosure are referred to as “interfering nucleic acids”. The interfering nucleic acids include, but are not limited to, small interfering RNA (siRNA), short hairpin RNA (shRNA), microRNA (miRNA), single-stranded RNA (ssRNA), double-stranded RNA (dsRNA), antisense oligonucleotide (ASO) and aptamer.

Small Interfering RNAs (siRNAs)

In one aspect, provided herein are compounds comprising a small interfering ribonucleic acid (siRNA), which reduce expression of the human SLC10A1 gene (hSLC10A1). In some embodiments, the compounds degrade mRNA transcripts of hSLC10A1. hSLC10A1 encodes the sodium taurocholate co-transporting polypeptide (NTCP) protein. NTCP is primarily expressed in the liver and mediates the uptake of bile acids into hepatocytes. An example of a bile acid is the bile acid taurocholic acid (TCA). Additionally, in HDV and HBV, NTCP serves as a cellular receptor for viral attachment and entry for infection of hepatocytes. In some embodiments, the siRNAs described herein targets hSLC10A1 mRNA and degrade hSLC10A1 mRNA via an RNA-induced silencing complex (RISC).

In some embodiments, the siRNA is from about 8 to about 50 nucleotides in length. In some embodiments, the siRNA molecule is from about 10 to about 50 nucleotides in length. In some instances, the siRNA molecule is from about 10 to about 30, from about 15 to about 30, from about 18 to about 25, form about 18 to about 24, from about 19 to about 23, or from about 20 to about 22 nucleotides in length. In some examples, the siRNA molecule comprises about 17, 18, 19, 20, 21, 22 or 23 nucleotides. In one embodiment, the siRNA comprises 19 nucleotides. In one embodiment, the siRNA comprises 21 nucleotides. In another example, the siRNA comprises 23 nucleotides.

In some embodiments, the siRNA is a RNA duplex of nucleotides formed by the complementary pairing between two regions of a RNA molecule, i.e., the sense strand and antisense strand of the siRNA. As a non-limiting example, the siRNA comprises a duplex comprising a sense strand of 19 nucleotides and an antisense strand of 21 nucleotides. In another example, the siRNA comprises a duplex comprising a sense strand of 21 nucleotides and an antisense strand of 23 nucleotides. In some cases, the sense and antisense strands are the siRNA are connected through a hairpin loop structure. In some embodiments, the siRNA targets a SLC10A1 mRNA transcript through the nucleotide sequence of the duplex portion of the siRNA that is complementary to a targeting sequence in the SLC10A1 mRNA transcript.

In some embodiments, the siRNA targeting hSLC10A1 mRNA comprises a sequence that is complementary to a portion of the nucleic acid sequence of the hSLC10A1 mRNA transcript (i.e., a target sequence). The siRNA molecule may have a nucleic acid sequence that is about 60%, 65%, 70%, 75%, 80%, 85%, 90%, 91%, 92%, 93%, 94%, 95%, 96%, 97%, 98%, 99% or 100% complementary to a portion of the nucleic acid sequence of hSLC10A1 mRNA. The siRNA sequence is complementary to a portion of the nucleic acid sequence of the human SLC10A1 mRNA transcript. In some embodiments, the siRNA sequence is complementary to a portion of the nucleic acid sequence encoding the amino acid sequence of SEQ ID NO: 11. In some embodiments, the sequence of the siRNA molecule is at least 70% complementary to the nucleic acid sequence encoding SEQ ID NO: 11. In some embodiments, the sequence of the siRNA molecule is at least 75% complementary to the nucleic acid sequence encoding SEQ ID NO: 11. In some embodiments, the sequence of the siRNA molecule is at least 80% complementary to the nucleic acid sequence encoding SEQ ID NO: 11. In some embodiments, the sequence of the siRNA molecule is at least 85% complementary to the nucleic acid sequence encoding SEQ ID NO: 11. In some embodiments, the sequence of the siRNA molecule is at least 90% complementary to the nucleic acid sequence encoding SEQ ID NO: 11. In some embodiments, the sequence of the siRNA molecule is at least 91% complementary to the nucleic acid sequence encoding SEQ ID NO: 11. In some embodiments, the sequence of the siRNA molecule is at least 92% complementary to the nucleic acid sequence encoding SEQ ID NO: 11. In some embodiments, the sequence of the siRNA molecule is at least 93% complementary to the nucleic acid sequence encoding SEQ ID NO: 11. In some embodiments, the sequence of the siRNA molecule is at least 94% complementary to the nucleic acid sequence encoding SEQ ID NO: 11. In some embodiments, the sequence of the siRNA molecule is at least 95% complementary to the nucleic acid sequence encoding SEQ ID NO: 11. In some embodiments, the sequence of the siRNA molecule is at least 96% complementary to the nucleic acid sequence encoding SEQ ID NO: 11. In some embodiments, the sequence of the siRNA molecule is at least 97% complementary to the nucleic acid sequence encoding SEQ ID NO: 11. In some embodiments, the sequence of the siRNA molecule is at least 98% complementary to the nucleic acid sequence encoding SEQ ID NO: 11. In some embodiments, the sequence of the siRNA molecule is at least 99% complementary to the nucleic acid sequence encoding SEQ ID NO: 11.

In some embodiments, the siRNA comprises a sequence that is complementary to a sequence of in the 3′ untranslated region (3′UTR), the coding region or the 5′ untranslated region (5′UTR) of a human SLC10A1 mRNA transcript. In some embodiments, the sequence of the siRNA molecule is 100% complementary to a portion of the nucleic acid sequence encoding SEQ ID NO: 11.

In some embodiments, the sequence of the siRNA molecule has 5 or less mismatches to a target sequence described herein. In some embodiments, the sequence of t the siRNA molecule has 4 or less mismatches to a target sequence described herein. In some instances, the sequence of the siRNA molecule has 3 or less mismatches to a target sequence described herein. In some cases, the sequence of the siRNA molecule has 2 or less mismatches to a target sequence described herein. In some cases, the sequence of the siRNA molecule has 1 or less mismatches to a target sequence described herein.

In some embodiments, the siRNA targeting a SLC10A1 mRNA transcript may comprise one or more chemical modifications; modification can be modifications of ribose (sugar), phosphates and/or nucleobases. Modifications can increase siRNA delivery, among other things, and stability (e.g., against nucleases) and/or reduce immune response.

The modification of the ribose moiety, e.g., substitutions in the 2′-position most effectively protect siRNAs against the action of serum nucleases, as the 2′OH group participates in the cleavage of RNA by endoribonucleases. The hydrogen of the 2′OH may be substituted by a methyl residue (2′-O-methyl modification; 2′-O′-Me), 2-O′-MOE, 2′-O-benzyl. The oxygen in some cases may be replaced by 2′-fluorine (2′F). In some embodiments, modifications at 2′ hydroxyl group of the ribose moiety may include an H, OR, R, halo, SH, SR, NH2, NHR, NR2, or CN, wherein R is an alkyl moiety. Exemplary alkyl moiety includes, but is not limited to halogens, sulfurs, thiols, thioethers, thioesters, amines (primary, secondary, or tertiary), amides, ethers, esters, alcohols and oxygen. In some embodiments, other positions in ribose, such as 4′ Carbon can be modified as well. Ribose modifications are not limited to substitutions in structure; nucleic acid analog with a modified structure of the furanose cycle, such as derivatives containing a-membered HNA, CeNA, and ANA and 7-membered rings, LNA, tricycle and acyclic derivatives. Those derivatives can protect siRNAs from the action of nucleases.

Modified nucleotides may include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In some examples, the modification at the 2′ hydroxyl group is a 2′-O-methyl (2′-O-Me) modification or a 2′-O-methoxyethyl (2′-O-MOE) modification.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, aminated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

In some embodiments, the modifications include nucleotide analogues. Nucleotide analogues further comprise morpholinos, peptide nucleic acids (PNAs), methylphosphonate nucleotides, thiolphosphonate nucleotides, 2′-fluoro N3-P5′-phosphoramidites, 5′-anhydrohexitol nucleic acids (HNAs), or a combination thereof.

In some embodiments, the siRNA comprises one or more of the artificial nucleotide analogues described herein. Exemplary artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the siRNA includes one or more backbone modifications. In some instances, the nucleotides are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

In some embodiments, one or more modifications optionally occur at the internucleotide linkage. In some embodiments, modified internucleotide linkage include, but is not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′linkage or 2′-5′linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof.

In some embodiments, the siRNA comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, from about 90% to about 100% modification, from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, from about 80% to about 90% modification, from about 10% to about 80% modification, from about 20% to about 80% modification, from about 30% to about 80% modification, from about 40% to about 80% modification, from about 50% to about 80% modification, from about 60% to about 80% modification, from about 70% to about 80% modification, from about 10% to about 70% modification, from about 20% to about 70% modification, from about 30% to about 70% modification, from about 40% to about 70% modification, from about 50% to about 70% modification, from about 60% to about 70% modification, from about 10% to about 60% modification, from about 20% to about 60% modification, from about 30% to about 60% modification, from about 40% to about 60% modification, from about 50% to about 60% modification, from about 10% to about 50% modification, from about 20% to about 50% modification, from about 30% to about 50% modification, from about 40% to about 50% modification, from about 10% to about 40% modification, from about 20% to about 40% modification, from about 30% to about 40% modification, from about 10% to about 30% modification, from about 20% to about 30% modification, and from about 10% to about 20% modification.

In some embodiments, the siRNA molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23 or more modifications.

In some embodiments, the siRNA molecule comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both.

In some embodiments, the siRNA is conjugated to one or more of N-acetyl-D-galactose (GalNAC), cholesterol, lipid, lipophilic molecule, polymer, peptide, ligand, or antibody.

In some embodiments, the siRNA is conjugated to one or more N-acetyl-D-galactose (GalNAc) residues. An siRNA that is conjugated to N-acetyl-D-galactose is referred to herein as a “GalNAc siRNA conjugate.” The following patent documents describe methods for conjugating nucleic acids like siRNAs to GalNAc: U.S. Pat. No. 8,575,123; U.S. Publication No. 2009/0239814; and U.S. Pat. No. 9,708,607. Each of the aforementioned patent documents is incorporated by reference herein in its entirety. The GalNAc siRNA conjugate of the disclosure may have any structure described in U.S. Pat. No. 8,575,123; U.S. Publication No. 2009/0239814; and U.S. Pat. No. 9,708,607, except that the nucleic acid sequence described in these patent documents is replaced by an siRNA described herein (e.g., SEQ. ID. No 3-6). Without being bound by theory, GalNAc siRNA conjugates bind to the asialoglycoprotein receptor (ASGPR). ASGPR is selectively expressed in hepatocytes. In some embodiments, GalNAc siRNA conjugates selectively enter hepatocytes and target hSLC10A1 mRNA transcripts therein. Binding of the siRNA to the hSLC10A1 mRNA triggers degradation of the hSLC10A1 mRNA via RISC.

In some embodiments, a linker is used to siRNA is conjugated to the one or more GalNAc. In some embodiments, the linker is a bivalent C₁-C₅₀ saturated or unsaturated, straight or branched alkyl, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heteroaryl, cycloalkyl, or cycloalkenyl.

In some embodiments, the linker is a non-cleavable linker. In other embodiments, the linker is a cleavable linker. The linker described herein can be a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

Provided herein are lipid nanoparticles (LNP) comprising any of the compounds described herein. In some embodiments, the lipid nanoparticle (LNP) allows delivery of an siRNA to the liver. U.S. Pat. No. 9,278,130 and US Publication No. 2013/0243848 describe siRNA LNPs and methods of manufacturing the same. These references are incorporated by reference herein in its entirety.

In some embodiments, the siRNA is conjugated with a peptide, for example, a targeting peptide to increase delivery to a site of interest (e.g., the liver). The peptide is conjugated to the 5′ terminus of the siRNA molecule, the 3′ terminus of the siRNA molecule, an internal site on the siRNA molecule, or in any combinations thereof.

In some embodiments, the siRNA is conjugated with a non-peptide ligand.

In some embodiments, the siRNA is conjugated to an antibody or fragment thereof. In some cases, the fragment is a binding fragment. Exemplary antibodies and fragments include but are not limited to, mAb, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, or a chemically modified derivative thereof.

In some embodiments, the siRNA is conjugated to a steroid. Exemplary steroids include cholesterol, phospholipids, di- and triacylglycerols, fatty acids, hydrocarbons that are saturated, unsaturated, comprise substitutions, or combinations thereof. In some instances, the siRNA is conjugated with cholesterol. In some embodiments, the siRNA is conjugated with a fatty acid. In some instances, cholesterol is conjugated by one or more of any known conjugation chemistry to the siRNA described herein.

In some embodiments, the siRNA is conjugated with a polymer, such as a natural or synthetic polymer. A polymer includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer comprises PEG.

In other embodiments the siRNA is conjugated with another nucleic acid molecule that does not hybridize to a target sequence or SLC10A1 mRNA, but instead for example, is capable of selectively binding to a cell surface marker.

In some embodiments, the compounds described herein are delivered to liver cells. In some embodiments, the liver cell is a hepatocyte, a hepatic stellate cell, a Kupffer cell, or a liver sinusoidal endothelial cell.

In some embodiments, provided herein are compositions containing any of the compounds or nanoparticles described herein.

The coding region of hSLCA1 is provided below as SEQ ID NO: 1: 5′-

(SEQ ID NO: 1)
ATGGAGGCCCACAACGCGTCTGCCCCATTCAACTTCACCCTGCCACCCA
ACTTTGGCAAGCGCCCCACAGACCTGGCACTGAGCGTCATCCTGGTGTT
CATGTTGTTCTTCATCATGCTCTCGCTGGGCTGCACCATGGAGTTCAGC
AAGATCAAGGCTCACTTATGGAAGCCTAAAGGGCTGGCCATCGCCCTGG
TGGCACAGTATGGCATCATGCCCCTCACGGCCTTTGTGCTGGGCAAGGT
CTTCCGGCTGAAGAACATTGAGGCACTGGCCATCTTGGTCTGTGGCTGC
TCACCTGGAGGGAACCTGTCCAATGTCTTCAGTCTGGCCATGAAGGGGG
ACATGAACCTCAGCATTGTGATGACCACCTGCTCCACCTTCTGTGCCCT
TGGCATGATGCCTCTCCTCCTGTACATCTACTCCAGGGGGATCTATGAT
GGGGACCTGAAGGACAAGGTGCCCTATAAAGGCATCGTGATATCACTGG
TCCTGGTTCTCATTCCTTGCACCATAGGGATCGTCCTCAAATCCAAACG
GCCACAATACATGCGCTATGTCATCAAGGGAGGGATGATCATCATTCTC
TTGTGCAGTGTGGCCGTCACAGTTCTCTCTGCCATCAATGTGGGGAAGA
GCATCATGTTTGCCATGACACCACTCTTGATTGCCACCTCCTCCCTGAT
GCCTTTTATTGGCTTTCTGCTGGGTTATGTTCTCTCTGCTCTCTTCTGC
CTCAATGGACGGTGCAGACGCACTGTCAGCATGGAGACTGGATGCCAAA
ATGTCCAACTCTGTTCCACCATCCTCAATGTGGCCTTTCCACCTGAAGT
CATTGGACCACTTTTCTTCTTTCCCCTCCTCTACATGATTTTCCAGCTT
GGAGAAGGGCTTCTCCTCATTGCCATATTTTGGTGCTATGAGAAATTCA
AGACTCCCAAGGATAAAACAAAAATGATCTACACAGCTGCCACAACTGA
AGAAACAATTCCAGGAGCTCTGGGAAATGGCACCTACAAAGGGGAGGAC
TGCTCCCCTTGCACAGCCTAG-3′.

The sequence of the 3′ untranslated region of hSLC10A1 (referred to herein as the “3′ UTR”) is provided herein as SEQ ID NO: 2:

(SEQ ID NO: 2)
5′-CCCTTCCCCTGGTGGCCTGGATTCTGGTCCCAAAGCAATTCTGAAA
GCCAGTGTGGTAAACTAGAGAGAGCAGCAAAAACACCAGTCTTGCCTGA
GTCTTTCTCCAGCATTTCCAGTACATCTATCAGAATCATCAAGTCTTGG
CCGGGAACACAGACAGGGTGTCTACCCAAGAAGCCTCACCTATCCCCAA
CTTAGAATTTGCTACTTATTTTAAAGACTTGTTCAGTGACTGTAAACTC
TATGAAACCAGAAACCGAATCTGCCTCTTGCTGGGATCTCTAAAAGTGT
CTGATAAGCATCTTAAAGTCACTCAATTCCTGAACTAATCAATATATAT
GTTTAACCCATTACTCAAATACCCAAATCCCATTCCAAGTTTTGTGACC
CAAAAGAGAAATAAATGCTCACAAGTGCTGTAGAATTAAACTTCAGAAG
TTCTAACCTAAAAAGTTCAGATCCTATTCCTTCCCTTTTGACATTATTG
GGATGATGCTCCCGAAAAGTCAAATTTGACATCAAGTATGCAAAAGTGA
ACACAGTAAGATGCAATCAGGCAAAACAAACTCAAAAAATAGCTAATGA
AATGAAAAAACTGGGCGAATGCATCATGTTAGTAGAGGAGGAAAACTTT
TGACAAGGAAAAACCAGGAAACAAACACATACATTAACACAATGTTACC
TCACTAATAATCTCTTTTTTAAGTTCAGTAGGTATTTGGTTTTAACACA
AGGCGCCCCCAAAACTGGGTACAGCAAACTACTGCCAAAATGAGTCGTT
TCCCAATTCAAAGAAAAATGTTCAAATACCTAGAATTCACAAATTTCAA
ATTGTCTCTAATAAAATTTAAACATTTTGCATATCA-3′.

In some embodiments, the siRNA sequence is complementary to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, nucleic acids in mRNA transcribed by SEQ ID NO. 1 or SEQ ID NO. 2. In some embodiments, the siRNA comprises a sequence about 60%, 65%, 70%, 75%, 80%, 85%, 90%, 91%, 92%, 93%, 94%, 95%, 96%, 97%, 98%, 99% or 100% complementary to a portion of the nucleic acid sequence of SEQ ID NO: 1. In some embodiments, the siRNA comprises a sequence about 60%, 65%, 70%, 75%, 80%, 85%, 90%, 91%, 92%, 93%, 94%, 95%, 96%, 97%, 98%, 99% or 100% complementary to a portion of the nucleic acid sequence of SEQ ID NO: 2.

In some embodiments, the siRNA used to reduce expression of hSLC10A1 is selected from any one of SEQ ID NOS: 3-6. Table 1 provides the nucleic acid sequences of SEQ ID NOS: 3-6.

TABLE 1
siRNAs that reduce expression of hSLC10A1

| siRNA | Sequence (5′ to 3′) | SEQ ID NO |
|---|---|---|
| #1 | UCCCCAACUUAGAAUUUGC | 3 |
| #2 | GCGUCAUCCUGGUGUUCAU | 4 |
| #3 | GCUUUCUGCUGGGUUAUGU | 5 |
| #4 | CUUUCCACCUGAAGUCAUU | 6 |

In some embodiments, the siRNA used to reduce expression of hSLC10A1 is SEQ ID NO: 3. SEQ ID NO: 3 binds to positions 187-205 of SEQ ID NO: 2. The nucleic acid sequence of positions 187-205 of SEQ ID NO: 2 is 5′-TCCCCAACTTAGAATTTGC-3′ (SEQ ID NO: 7).

In some embodiments, the siRNA used to reduce expression of hSLC10A1 is SEQ ID NO: 4. SEQ ID NO: 4 binds to positions 83-101 of SEQ ID NO: 1. The nucleic acid sequence of positions 83-101 of SEQ ID NO: 1 is 5′-GCGTCATCCTGGTGTTCAT-3′ (SEQ ID NO: 8).

In some embodiments, the siRNA used to reduce expression of hSLC10A1 is SEQ ID NO: 5. SEQ ID NO: 5 binds to positions 698-716 of SEQ ID NO: 1. The nucleic acid sequence of positions 698-716 of SEQ ID NO: 1 is 5′-GCTTTCTGCTGGGTTATGT-3′ (SEQ ID NO: 9).

In some embodiments, the siRNA used to reduce expression of hSLC10A1 is SEQ ID NO: 6. SEQ ID NO: 6 binds to positions 819-837 of SEQ ID NO: 1. The nucleic acid sequence of positions 819-837 of SEQ ID NO: 1 is 5′-CTTTCCACCTGAAGTCATT-3′ (SEQ ID NO: 10).

In some embodiments, the siRNA comprises a sequence having at least 70%, 75%, 80%, 85%, 90%, 95%, 96%, 97%, 98%, 99%, or 100% sequence identity to a sequence selected from SEQ ID NOs: 3-6.

In some embodiments, the siRNA has reduced off-target effect. As used herein, “off-target” or “off-target effects” refer to any instance in which a nucleic acid molecule discussed herein against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleotide molecule.

Nucleotide synthesis methods for the preparation of short RNA sequences are known to those skilled in the art and described in the prior art. The siRNAs of the present disclosure may be produced by chemical synthesis, and are represented by duplexes of small oligonucleotides.

### Other Interfering Nucleic Acid Molecules Targeting SLC10A1

In addition to siRNA molecules, other interfering nucleic acid molecules can interact with a target mRNA and silence gene expression. In accordance, human SLC10A1 mRNA transcript may be repressed using other types of small nucleic acid molecules, including but not limited to short hairpin RNAs (shRNAs), dsRNAs, miRNAs, antisense oligonucleotides, and aptamers. Those interfering nucleic acids are defined as agents which function to inhibit expression of a target gene. These are the effector molecules for inducing RNAi, leading to posttranscriptional gene silencing with RNA-induced silencing complex (RISC). In the case of antisense oligonucleotides (ASO's) the mechanism of silencing can be via direct hybridization with the target mRNA in a complementary manner resulting in degradation by a cellular RNase H enzyme. The ASO may be a short DNA or RNA oligomer molecule that is delivered.

An “shRNA molecule” includes a conventional stem-loop shRNA, which forms a precursor miRNA (pre-miRNA) that can silence expression of a target gene. S ingle-stranded interfering RNA has been found to effect mRNA silencing. Single-stranded interfering RNAs can be synthesized chemically or by in vitro transcription or expressed endogenously from vectors or expression cassettes as described herein in reference to double-stranded interfering RNAs. An antisense oligonucleotide refers to a nucleic acid (in preferred embodiments, an RNA) (or analog thereof), having sufficient sequence complementarity to a target RNA (i.e., the RNA for which splice site selection is modulated) to block a region of a target RNA (e.g., SLC10A1 mRNA) in an effective manner.

### IV. Pharmaceutical Compositions

In accordance, the present disclosure provides compositions, among other things, comprising at least one interfering nucleic acid targeting a human SLC10A1 mRNA transcript. In particular, the composition comprises an siRNA described herein.

In some embodiments, a pharmaceutical composition comprising an siRNA described herein as active ingredient is provided; the pharmaceutical composition further comprises one or more pharmaceutically acceptable carriers. The carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, the carriers include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the carriers include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the carriers include diluents which are used to stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

Lubricants and glidants are also optionally included in the pharmaceutical compositions described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Other exemplary carriers may be included in the pharmaceutical compositions described herein include plasticizers, disintegration agents or disintegrants to facilitate the breakup or disintegration of a substance, and filling agents (e.g., lactose, calcium carbonate, calcium phosphate, dibasic calcium phosphate calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like), solubilizers and stabilizers.

### V. Formulations and Administration

In some embodiments, compounds comprising an interfering nucleic acid (e.g., siRNA), compositions comprising the same may be delivered in solution, in suspension, or in bioerodible or non-bioerodible delivery devices. The compounds can be delivered alone or as components of defined, covalent conjugates. The compounds can also be complexed with cationic lipids, cationic peptides, or cationic polymers; complexed with proteins, fusion proteins, or protein domains with nucleic acid binding properties (e.g., protamine); or encapsulated in nanoparticles or liposomes. Tissue- or cell-specific delivery can be accomplished by the inclusion of an appropriate targeting moiety such as an antibody or antibody fragment.

In some embodiments, the pharmaceutical formulations described herein are administered to a patient in need by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for subcutaneous administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

As a non-limiting example, the pharmaceutical formulation is a lipid nanoparticle (LNP).

In some embodiments, the compositions of the present disclosure are prepared in a solid lyophilized form that can be stored and reconstituted to provide a formulation for administration of treatment. Any process for preparing a solid lyophilized drug product known in the art may be used to prepare the lyophilized products described herein (e.g., U.S. Pat. No. 10,300,018). In some aspects, the lyophilized products are stored until ready to be administration. For example, the lyophilized products comprising a nucleic acid therapeutic described herein can be stored at 4° C. and/or −20° C. In some embodiments, the solid lyophilized products can be reconstituted in sterile water to form a syringible formulation for administration.

Accordingly, the present disclosure further contemplates formulations made by reconstitution of the solid lyophilized products.

In some embodiments, the pharmaceutical formulation comprises a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus (AAV), retrovirus, adenovirus, or alphavirus.

The dose of the compounds described herein and/or second pharmaceutical agents described herein depends on the specific compound, and on the specific condition to be treated.

### VI. Methods of Uses Thereof

In accordance with the present disclosure, the siRNA molecules, conjugates thereof and pharmaceutical compositions described herein are administered for therapeutic applications, for example, for treating diseases and/or disorders that are associated with NTCP activities. In some embodiments, the disease is a cholestatic disorder, hepatitis B, hepatitis D, nonalcoholic fatty liver disorder (NAFLD) and NASH.

In some embodiments, provided herein are methods of knocking out NTCP comprising administering a compound and/or a composition described herein. In some embodiments, knocking out NTCP refers to reducing translation of NTCP. In some embodiments, knocking out NTCP refers to reducing production of NTCP. In some embodiments, knocking out NTCP refers to degrading mRNA transcripts of the hSLC10A1 gene. In some embodiments, the compound is administered to a cell. In some embodiments, the compound is administered to a patient. In some embodiments, the compound is administered to a pediatric patient. In some embodiments, the compound is administered to an adult.

In some embodiments, provided herein are methods of treating a cholestatic disorder in a patient in need thereof comprising administering a compound, and/or a composition described herein. In some embodiments, the cholestatic disorder is associated with a bile acid transporter (i.e., NTCP). In some embodiments, the cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), intrahepatic cholestasis of pregnancy (ICP), ductal plate abnormalities, Caroli syndrome, and bile acid synthesis defects.

In some embodiments, provided herein is a method of treating PFIC in a patient in need thereof comprising administering a compound and/or a composition described herein. PFIC is a pediatric disorder caused by mutations in transporters that control bile flow. PFIC occurs within the first three months of life. There are four types of PFIC: type 1, type 2, type 3, or type 4. Type 1 and Type 2 PFIC are most common. In some embodiments, the methods described herein treat Type 1, Type 2, Type 3, or Type 4 PFIC.

In some embodiments, provided herein is a method of treating ALGS in a patient in need thereof comprising administering a compound of Section II. ALGS is a pediatric disorder caused by mutations in the NOTCH2 and JAG1 genes which result in narrow, malformed, or deficient bile ducts. In some embodiments, a patient with ALGS has a loss of function mutation in JAG1 or NOTCH2. Patients with ALGS exhibit cholestasis and multisystem problems. In some embodiments, patients experience early onset ALGS. Early onset ALGS may occur during infancy.

In some embodiments, provided herein is a method of treating BA in a patient in need thereof comprising administering a compound and/or a composition described herein. BA is a pediatric disorder caused by neuro-inflammatory destructions of intra- or extra-hepatic bile ducts in infants. BA typically occurs between 2 to 8 weeks after birth. Currently, BA is fatal without a Kasai procedure. BA is the number one cause of pediatric liver transplant.

In some embodiments, provided herein is a method of treating PBC in a patient in need thereof comprising administering a compound and/or a composition described herein. PBC is a disorder characterized by T cell mediated destruction of small bile duct epithelial cells, which causes ductopenia. PBC occurs in adults and more commonly in females. In some embodiments, provided herein is a method of treating a female patient with PBC. The typical onset of PBC is between ages 40 and 60.

In some embodiments, provided herein is a method of treating PSC in a patient in need thereof comprising administering a compound and/or a composition described herein. PSC is an immune-mediated chronic debilitating disorder that affects adults. PSC is more common in men. In some embodiments, provided herein is a method of treating a male patient with PSC. The typical onset of PSC is at or after age 40.

In some embodiments, provided herein is a method of treating ICP in a patient in need thereof comprising administering a compound and/or a composition described herein. ICP involves a combination of genetic susceptibility, hormonal, and environmental factors. In some embodiments, provided herein is a method of treating a pregnant female patient with ICP.

In some embodiments, provided herein is a method of treating hepatitis D and/or hepatitis B in a patient in need thereof comprising administering a compound and/or a composition described herein. Hepatitis B is a liver infection caused by the hepatitis B virus (HBV). Hepatitis D is a liver disease resulting from co-infection or superinfection with the hepatitis D virus (HDV) with HBV. The disease pathology resulting from a HDV and HBV infection can be extremely serious leading to severe complications and a greater likelihood rapid progression to cirrhosis and liver cancer. Patients with hepatitis D experience jaundice, joint pain, abdominal pain, vomiting, dark urine, and fatigue.

In some embodiments, provided herein is a method of treating CHF comprising administering a compound and/or a composition described herein. CHF is a genetic disorder that affects the liver and kidneys. CHF is caused by abnormal development of the portal veins and bile ducts that begins with a malformation in the embryonic structure called the ductal plate.

In some embodiments, provided herein is a method of treating nonalcoholic fatty liver disease (NAFLD) comprising administering a compound and/or a composition described herein. NAFLD is a condition in which fat builds up in a patient's liver. NAFLD is one of the most common cause of liver disease in the United States.

In some embodiments, provided herein is a method of treating non-alcoholic steatohepatitis (NASH) comprising administering a compound and/or a composition described herein. NASH is an advanced form of NAFLD. In addition to buildup of fat in the liver, NASH patients have inflammation and damage that leads to scarring of the liver. Scarring of the liver may lead to cirrhosis and permanent damage of the liver.

In some embodiments, the methods described herein cause a decrease in the amount of hSLC10A1 mRNA transcripts compared to before administration of a compound described herein. In some embodiments, the amount of hSLC10A1 mRNA transcripts decreases by from about 25% to about 100% compared to the amount of hSLC10A1 mRNA transcripts before administration of a compound described herein. For example, the amount of hSLC10A1 mRNA transcripts may decrease by about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% compared to before administration of a compound described herein. In some embodiments, the amount of hSLC10A1 mRNA transcripts decreases by from about 25% to about 100% for about 1 day, about 2 days, about 3 days, about 4 days, about 5 days, about 6 days, about 7 days (1 week), about 8 days, about 9 days, about 10 days, about 11 days, about 12 days, about 13 days, about 14 days (2 weeks), about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1 month, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1 year, about 18 months, about 2 years, or more, compared to the amount of hSLC10A1 mRNA transcripts before administration of a compound described herein at a given time point.

In some embodiments, translation of hSLC10A1 mRNA transcripts decreases by from about 25% to about 100% compared to translation of hSLC10A1 mRNA transcripts before administration of a compound described herein. For example, the translation of hSLC10A1 mRNA transcripts may decrease by about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% compared to before administration of a compound described herein.

In some embodiments, translation of hSLC10A1 mRNA transcripts decreases by from about 25% to about 100% for about 1 day, about 2 days, about 3 days, about 4 days, about 5 days, about 6 days, about 7 days (1 week), about 8 days, about 9 days, about 10 days, about 11 days, about 12 days, about 13 days, about 14 days (2 weeks), about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1 month, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1 year, about 18 months, about 2 years, or more, compared to translation of hSLC10A1 mRNA transcripts before administration of a compound described herein at a given time point.

In some embodiments, the methods provided herein attenuate NTCP mediated activities including but not limited to bile acid uptake in the liver, and HBV and/or HDV interaction.

### Combination Therapies

In some embodiments, the present compounds, composition and methods can be used in combination with other active ingredients. The term “in combination” means either a co-therapy or combination therapy, or a co-formulation in a single pharmaceutical form, or in a single commercial package, for example a kit or a blister of two or more active ingredients.

In one aspect, another therapy that targets the bile acid metabolism pathway may be used in combination with the present compound, compositions and/or methods for treating a cholestatic disorder.

In another aspect, the present compound, compositions and methods as described herein may be used in combination with one or more anti-viral therapies for treating hepatitis B and/or hepatitis D.

In some embodiments, the patient in need may further receive an anti-viral therapy for treating hepatitis B and/or hepatitis D. Exemplary antiviral medications for hepatitis B include entecavir, tenofovir, lamivudine, adefovir and telbivudine.

In some embodiments, the patient in need may be further treated with an immunomodulatory therapy. Exemplary immunomodulatory therapies include pegylated Interferon alfa-2b (Intron A), PD-1, PD-L1 or other immune checkpoint inhibitors, TLR agonists or other general or specific immunomodulatory agents.

In yet another aspect, the present compound, compositions and methods as described herein may be used in combination with another therapy of NAFLAD and NASH.

In some embodiments, two or more compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition.

### VII. Kits

In certain embodiments, the present invention also provides a kit that includes reagents for repressing the expression of SLC10A1 mRNA as cited herein in a cell. The kit may also contain positive and negative control siRNAs (e.g., a non-targeting control siRNA or an siRNA that targets an unrelated mRNA). The kit also may contain reagents for assessing knockdown of the intended target gene SLC10A1 (e.g., primers and probes for quantitative PCR to detect the target mRNA and/or antibodies against the corresponding protein for western blots). Alternatively, the kit may comprise an siRNA sequence and the instructions and materials necessary to generate the siRNA by in vitro transcription.

In some embodiments, a pharmaceutical combination in kit form is further provided that includes, in packaged combination, a carrier means adapted to receive a container means in close confinement therewith and a first container means including an interfering RNA composition and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### EXAMPLES

The following examples are provided to illustrate the disclosure and are merely for illustrative purpose only and should not be construed to limit the scope of the disclosure.

### Example 1: Knock Out of NTCP with an siRNA Targeting the SLC10A1 Gene

Purpose: The ability of a siRNA to knock out SLC10A1 was evaluated. Specifically, the siRNA, siRNA #4, was evaluated. siRNA #4 has the sequence 5′-CUUUCCACCUGAAGUCAUU-3′ (SEQ ID NO: 6). This siRNA binds to positions 819-837 of the coding region of SLC10A1 (SEQ ID NO: 1). SLC10A1 encodes for the protein NTCP, which transports bile acids like taurocholic acid (TCA) from outside of a cell to inside of a cell.

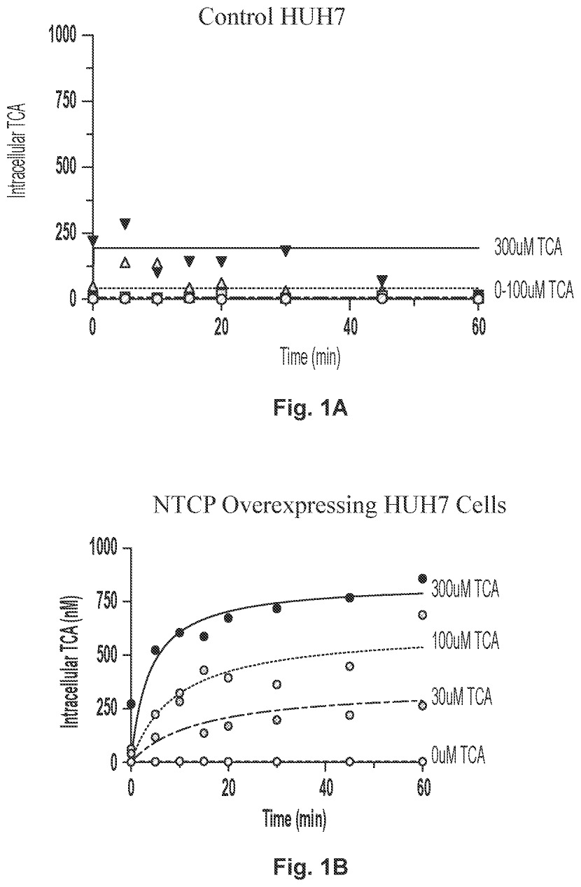

Methods and Results: The intracellular concentration of TCA was measured in control HUH7 cells and HUH7 cells overexpressing the SLC10A1 gene, which encodes for NTCP. HUH7 cells are human liver cells. FIG. 1A shows that control HUH7 cells (labeled “Control HUH7 cells”) having minimal native expression levels of SLC10A1, do not transport TCA into the cell after exposure of TCA at concentrations ranging from 0 to 300 μM. In contrast, HUH7 cells overexpressing SLC10A1 (labeled “NTCP Overexpressing HUH7 Cells”) transport TCA into the cell after exposure of the cells to 30 μM, 100 μM, and 300 μM TCA (FIG. 1B). Uptake of TCA was concentration and time-dependent.

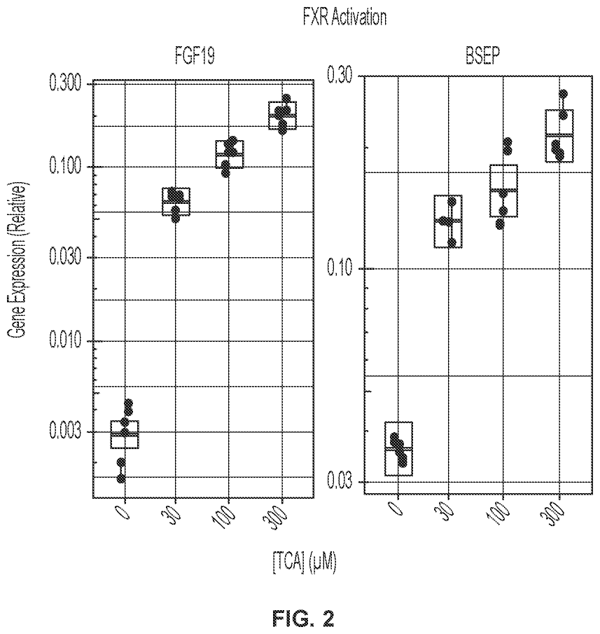

Intracellular TCA uptake regulates Farnesoid X receptor signaling (FXR) in primary hepatocytes. FXR activation leads to increased expression of FGF19 and BSEP. FGF19 encodes for the protein fibroblast growth factor 19, which regulates bile acid synthesis. BSEP encodes for the protein bile salt export pump, which is an efflux pump that eliminates substrates from hepatocytes. FIG. 2 shows increased gene expression of FGF19 and BSEP in primary human hepatocytes after exposure to 0 μM, 30 μM, 100 μM, or 300 μM TCA.

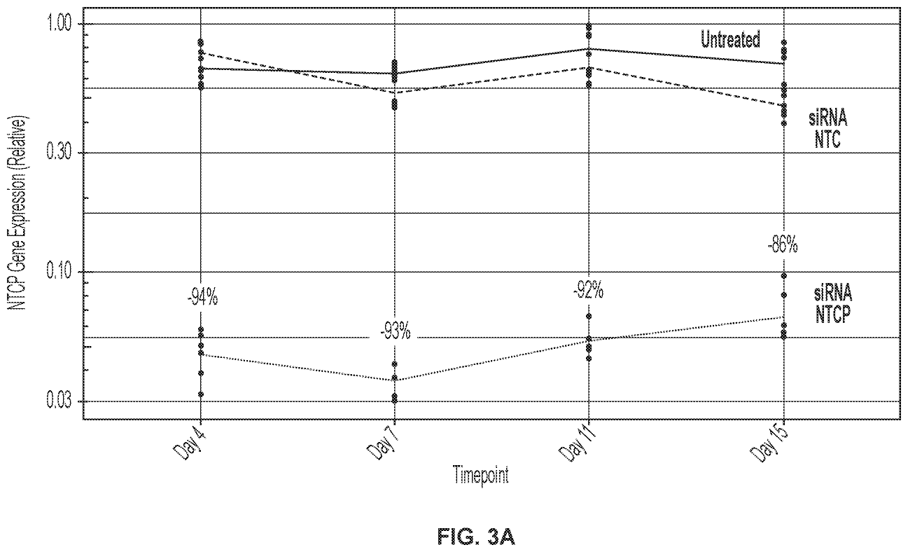

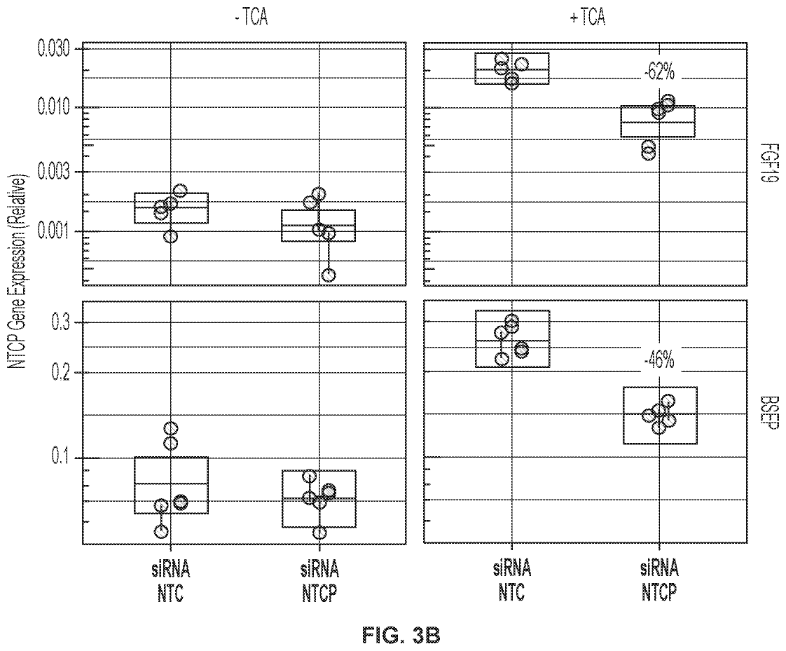

Treatment of primary human hepatocytes with endogenous expression of SLC10A1 with a single treatment of siRNA #4 resulted in reduced expression of SLC10A1 (labeled “NTCP”) for up to two weeks (FIG. 3A). In contrast, untreated cells and cells treated with a non-targeting control siRNA (SEQ ID NO: 11) that does not bind to the SLC10A1 (labeled “NTC #1) retained expression of SLC10A1. Additionally, siRNA knockdown of SLC10A1 leads to a reduction of the intracellular bile acid surrogates (FGF19, BSEP gene expression) in primary human hepatocytes (FIG. 3B). This data shows the potential of siRNAs for knocking down SLC10A1.

Future Experiments will evaluate the ability of alternative siRNAs (e.g., siRNA #1, siRNA #2, siRNA #3, or siRNA #4 of Table 1 having sequences of SEQ ID NO: 3, SEQ ID NO: 4, and SEQ ID NO: 5, respectively) to knock down SLC10A1.

Future Experiments will evaluate the ability of GalNAc siRNA conjugates containing siRNAs having sequences of SEQ ID NO: 3, SEQ ID NO: 4, or SEQ ID NO: 5 to knock down SLC10A1.

### Example 2: Reducing SLC10A1 Expression and Activity by Direct Uptake of siRNAs Targeting SLC10A1 Gene

This example shows that siRNAs #1-4 of Table 1 (SEQ ID Nos: 3-6) ((Horizon Discovery, catalogue A-007376) can reduce SLC10A1 gene expression and protein activity.

In this example, Cholesterol conjugated SLC10A1 siRNAs were evaluated in primary human hepatocytes up to 4 uM with 3 biological replicates. Primary human hepatocytes were isolated from a normal human liver, cryopreserved, and stored in liquid nitrogen until ready for experimental plating. Primary hepatocytes were thawed and plated on a collagen layer at 220,000 cells/cm² and treated with 0-4 uM siRNAs (#1-4) prior to hepatocyte attachment. The presence of the cholesterol moiety facilitates uptake in primary human hepatocytes. After plating, cells were maintained in a humidified incubator at 37° C. and 5% CO₂ for the remainder of the experiment. The cultures were in maintenance medium (DMEM/F-12 supplemented with 10% fetal bovine serum, 50 mg/ml gentamycin, 0.2% ITS (Fisher/MediaTech MT-25e800CR), and dexamethasone (Sigma Aldrich D4902; 1 mM at plating and 250 nM thereafter). 72 hours after initial treatment with the siRNAs, samples were collected for assessing gene knockdown or tested for bile acid uptake.

2.1 Quantification of mRNA Knockdown

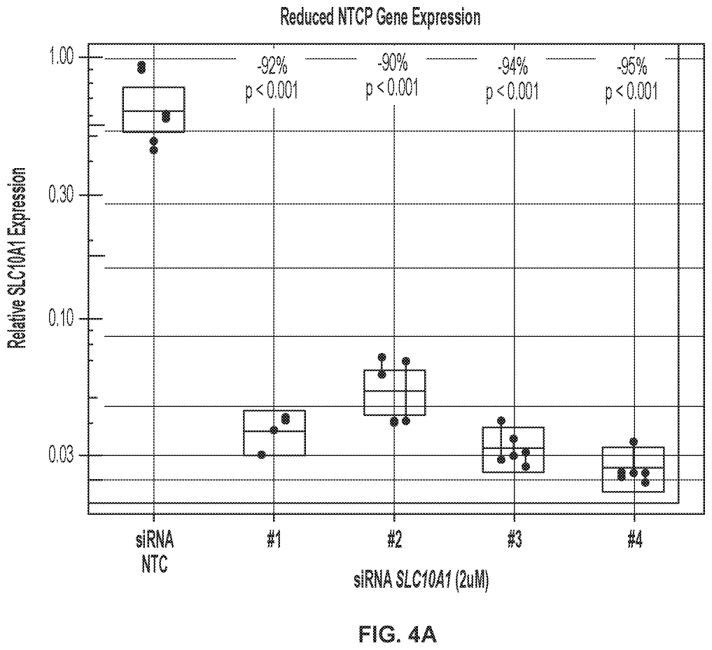

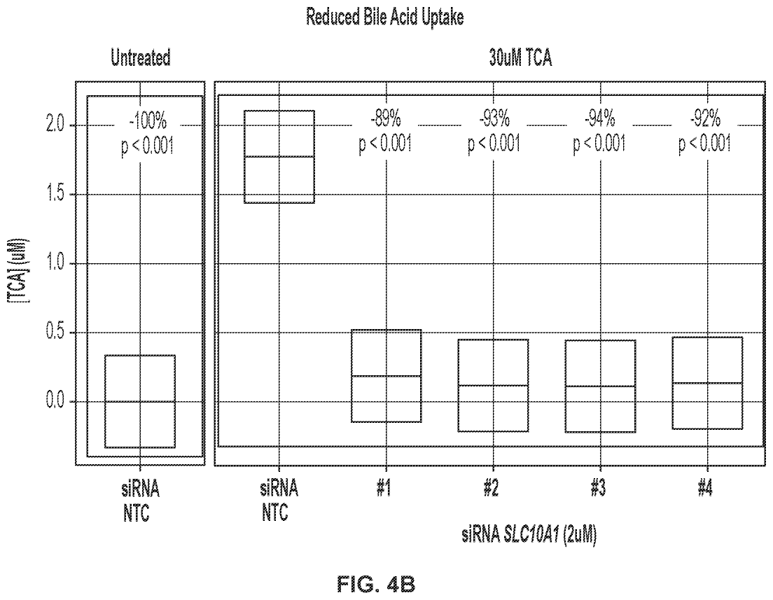

For assessing gene knockdown, hepatocytes' RNA was collected in trizol using manufacturer protocols. RNA was isolated using the Invitrogen Purelink RNA Mini kit (12183018A) according to manufacturer's instructions. Measurement of SLC10A1 mRNA levels utilized a real-time reverse transcriptase polymerase chain reaction (RT-PCR) in duplicate. RNA was multiplexed with probes specific for human SLC10A1 and/or a human RPS11 using a BioRad CFX96 PCR Machine. For determining the percent gene knockdown by the cholesterol-siRNAs, cycle times for SLC10A1 were normalized to RPS11 and then presented as fold change to the negative, non-targeting control at the same siRNA concentration. FIG. 4A shows the percent knockdown in primary hepatocytes using 4 unique siRNAs #1-4.

### 2.2 Quantification of Bile Acid Uptake

To evaluate the impact of siRNA knockdown on bile acid transport, hepatocytes were further treated with 30 uM of the bile acid, taurocholic acid (TCA), for 15 minutes. After this period, the cells were thoroughly washed in PBS and then lysed in acetonitrile to collect intracellular levels of TCA. Samples were processed and TCA was quantified using a RapidFire mass spectrometer. TCA concentrations were determined using a standard curve of TCA and statistical analysis was compared to the non-targeting control. In FIG. 4B shows the percent inhibition of bile acid transport in primary hepatocytes using 4 unique siRNAs #1-4.

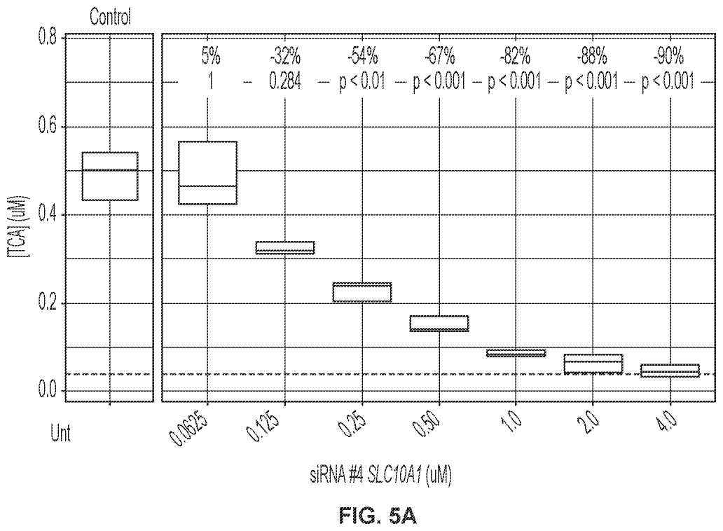

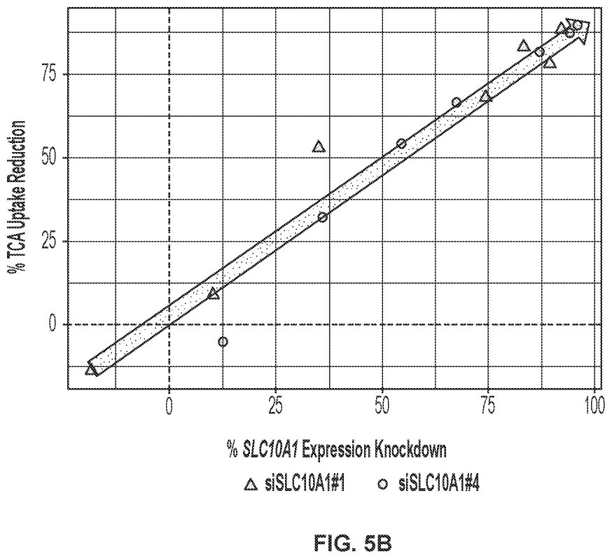

The reduction of TCA transport activity, as shown in FIG. 5A is siRNA concentration dependent. The results also demonstrate that the percent inhibition of TCA transport by siRNA is highly correlated to degree of gene knockdown observed (FIG. 5B).

### EQUIVALENTS AND SCOPES

Those skilled in the art would recognize, or be able to ascertain using no more than routine experimentation, many equivalents to the specific embodiments of the invention described herein. The scope of the invention is not intended to be limited to the above Description, but rather is as set forth in the following claims:

1. A compound comprising a small interfering ribonucleic acid sequence (siRNA) which represses translation of a human SLC10A1 mRNA transcript.

2. The compound of claim 1, wherein the siRNA comprises a nucleic acid sequence at least 80%, at least 85%, at least 90%, at least 95%, at least 96%, at least 97%, at least 98%, at least 99%, or 100% complementary to a portion of the nucleic acid sequence of the human SLC10A1 mRNA transcript.

3. The compound of claim 2, wherein the siRNA is complementary to a portion of the nucleic acid sequence of the human SLC10A1 mRNA transcript, or wherein the siRNA is complementary to a portion of the nucleic acid sequence encoding the amino acid sequence of SEQ ID NO: 11.

4. The compound of any one of claims 1-3, wherein the portion of the human SLC10A1 mRNA transcript locates in the 3′ end untranslated region (3′UTR), the coding region, and/or the 5′ end UTR region of the human SLC10A1 mRNA transcript.

5. The compound of claim 4, wherein the siRNA comprises a sequence complementary to a portion of the nucleic acid sequence of SEQ ID NO: 1, or to a portion of the nucleic acid sequence of SEQ ID NO: 2.

6. The compound of any one of the preceding claims, wherein the siRNA comprises about 12-30 nucleotides, or about 17-23 nucleotides.

7. The compound of claim 6, wherein the siRNA comprises 17, 18, 19, 20, 21, 22, 23, 24, or 25 nucleotides.

8. The compound of claim 7, wherein the siRNA comprises 19 nucleotides.

9. The compound of claim 7, wherein the siRNA comprises 21 nucleotides.

10. The compound of claim 7, wherein the siRNA comprises 23 nucleotides.

11. The compound of any one of claims 6-10, wherein the siRNA is a RNA duplex comprising a sense strand of 19 nucleotides and an antisense strand of 21 nucleotides.

12. The compound of any one of claims 6-10, wherein the siRNA is a RNA duplex comprising a sense strand of 21 nucleotides and an antisense strand of 23 nucleotides.

13. The compound of any one of the preceding claims, wherein the siRNA comprises at least one modification.

14. The compound of claim 13, wherein the modification comprises a sugar modification, a backbone modification and/or a nucleobase modification.

15. The compound of any one of the preceding claims, wherein the compound is conjugated to one or more of N-acetyl-D-galactose (GalNAC), cholesterol, lipid, lipophilic molecule, polymer, peptide, ligand, or antibody.

16. The compound of claim 15, wherein the siRNA is conjugated to one or more N-acetyl-D-galactose.

17. The compound of any one of the preceding claims, wherein the siRNA binds to the 3′ untranslated region of human SLC10A1.

18. The compound of any one of claims 1-16, wherein the siRNA binds to the coding region of human SLC10A1.

19. The compound of any one of the preceding claims, wherein the compound specifically targets human SLC10A1 mRNA transcripts of the liver.

20. The compound of claim 19, wherein the human SLC10A1 mRNA transcripts are located in a cell selected from the group consisting of hepatocytes, hepatic stellate cells, Kupffer cells, and liver sinusoidal endothelial cells.

21. The compound of any one of the preceding claims, wherein the siRNA comprises the sequence of any one of SEQ ID NOs: 3-6.

22. A method for degrading a human SLC10A1 mRNA transcript in a cell comprising administering the compound of any one of claims 1-21 to the cell.

23. The method of claim 22, wherein at least 50% of human SLC10A1 mRNA transcripts are degraded for at least 1 week.

24. The method of claim 22, wherein at least 90% of human SLC10A1 mRNA transcripts are degraded for at least 1 week.

25. The method of claim 22, wherein at least 95% of human SLC10A1 mRNA transcripts are degraded for at least 1 week.

26. The method of claim 22, wherein at least 98% of human SLC10A1 mRNA transcripts are degraded for at least 1 week.

27. The method of claim 22, wherein at least 50% of human SLC10A1 mRNA transcripts are degraded for at least 2 weeks.

28. The method of claim 22, wherein at least 90% of human SLC10A1 mRNA transcripts are degraded for at least 2 weeks.

29. The method of claim 22, wherein at least 95% of human SLC10A1 mRNA transcripts are degraded for at least 2 weeks.

30. The method of claim 22, wherein at least 98% of human SLC10A1 mRNA transcripts are degraded for at least 2 weeks.

31. The method of any one of claims 22-30, wherein the cell is a liver cell.

32. The method of claim 31, wherein the liver cell is a hepatocyte, a hepatic stellate cell, a Kupffer cell, or a liver sinusoidal endothelial cell.

33. A method of treating a cholestatic disorder in a patient in need thereof comprising administering to the patient a composition comprising a nucleic acid molecule that inhibits or reduces expression of Na⁺-taurocholate cotransporting polypeptide (NTCP).

34. The method of claim 33, wherein the cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP).

35. The method of claim 33 or 34, wherein after treating the patient exhibits reduced intrahepatic accumulation of bile acids.

36. The method of any one of claims 33-35, wherein after treating, the patient experiences an improvement in at least one symptom of a cholestatic disorder, selected from the group consisting of pruritis, mitochondrial damage and inflammation in the liver, and hepatic injury.

37. The method of any one of claims 32-36, wherein the nucleic acid molecule represses translation of a human SLC10A1 mRNA transcript, thereby silencing, or down-regulating expression of the human SLC10A1 mRNA transcript.

38. The method of any one of claims 32-37, wherein the nucleic acid molecule is a siRNA, a shRNA, a dsRNA, an antisense oligonucleotide (ASO), miRNA, or aptamer.

39. The method of claims 32-38, wherein the nucleic acid molecule comprises at least one modification.

40. The method of any one of claims 32-39, wherein the nucleic acid molecule is a siRNA.

41. The method of claim 40, wherein the siRNA comprises a nucleic acid sequence that is at least 80%, or at least 85%, or at least 90%, or at least 95%, or at least 96%, or at least 97%, or at least 98%, at least 99%, or 100% complementary to a portion of the nucleic acid sequence of the human SLC10A1 mRNA transcript.

42. The method of any one of claims 40-41, wherein the siRNA comprises about 12-30 nucleotides, or about 17-23 nucleotides.

43. The method of claim 42, wherein the siRNA comprises about 17, 18, 19, 20, 21, 22, 23, 24 or 25 nucleotides.

44. The method of claim 43, wherein the siRNA is a RNA duplex comprising a sense strand of 19 nucleotides and an antisense strand of 21 nucleotides.

45. The method of claim 43, wherein the siRNA is a RNA duplex comprising a sense strand of 21 nucleotides and an antisense strand of 23 nucleotides.

46. The method of any one of claims 40-45, wherein the siRNA comprises at least one sugar modification, nucleobase modification and/or a backbone modification.

47. The method of any one of claims 40-46, wherein the siRNA is conjugated to one or more of N-acetyl-D-galactose (GalNAC), cholesterol, lipid, lipophilic molecule, polymer, peptide, ligand, and antibody.

48. The method of claim 47, wherein the siRNA is conjugated with at least one GalNAc moiety, or at least one tri-antennary GalNAc moiety.

49. A method of treating a disease that is associated with bile acid transporter, NTCP, comprising silencing or downregulating a human SLC10A1 mRNA transcript with a siRNA that represses expression of the human SLC10A1 mRNA transcript.

50. The method of claim 46, wherein the disease is a cholestatic disorder, hepatitis B, hepatitis D, NAFLD or NASH.

51. The method of claim 50, wherein the disease is a cholestatic disorder.

52. The method of claim 51, wherein the NTCP mediated bile acid uptake is blocked.

53. The method of any one of claims 51-52, wherein the bile acid is reduced in the liver.

54. The method of any one of claims 51-53, wherein one of more symptoms of a cholestatic disorder is improved, selected from the group consisting of pruritis, mitochondrial damage and inflammation in the liver, and hepatic injury.

55. The method of claim 50, wherein the disease is hepatitis B or hepatitis D.

56. The method of claim 55, wherein the NTCP mediated Hepatitis B virus (HBV) interaction and/or Hepatitis D virus (HDV) interaction is prevented.

57. The method of claim 50, wherein the disease is Nonalcoholic fatty liver disease (NAFLD) or Non-Alcoholic Steatohepatitis (NASH).

58. The method of claim 57, wherein after treating, the patient experiences an improvement in at least one symptom of NAFLD or NASH, selected from the group consisting of fatty acid metabolism, inflammation and fibrosis.

59. A method of treating hepatitis D in a patient in need thereof comprising administering the compound of any one of claims 1-21.

60. A method of treating hepatitis B in a patient in need thereof comprising administering the compound of any one of claims 1-21.

61. A method of treating NAFLD or NASH in a patient in need thereof comprising administering the compound of any one of claims 1-21.

62. A lipid nanoparticle comprising the compound of any one of claims 1-21.

63. A composition comprising the compound of any one of claims 1-21, or the lipid nanoparticle of claim 62.

64. A pharmaceutical composition comprising the compound of any one of claims 1-21, or the lipid nanoparticle of claim 62, and a pharmaceutically acceptable carrier.

65. A method of treating a cholestatic disorder in a patient in need thereof comprising administering the composition of claim 63 or the pharmaceutical composition of claim 64.

66. The method of claim 63, wherein the cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP).

67. The method of claim 65 or 66, wherein after treating the patient exhibits reduced intrahepatic accumulation of bile acids.

68. The method of any one of claims 65-67, wherein after treating, the patient experiences an improvement in at least one symptom of a cholestatic disorder, selected from the group consisting of pruritis, mitochondrial damage and inflammation in the liver, and hepatic injury.

69. A method of treating hepatitis D or hepatitis B in a patient in need thereof comprising administering the composition of claim 63 or the pharmaceutical composition of claim 64.

70. The method of claim 69, further comprising administering to the patient with an anti-viral agent, an immunomodulatory agent or the combination thereof.

71. A method of treating NAFLD or NASH in a patient in need thereof comprising administering the composition of claim 63 or the pharmaceutical composition of claim 64.

72. The method of any one of claims 22-61, and 63-71, wherein the compound or the composition is administered subcutaneously, intramuscularly, or intravenously.

73. A method for blocking Na⁺-taurocholate cotransporting polypeptide (NTCP) mediated activities in the liver of a subject in need comprising administering to the subject with a composition comprising a nucleic acid molecule that targets a human SLC10A1 mRNA transcript in the liver, wherein the nucleic acid molecule inhibits expression of NTCP in the liver.

74. The method of claim 73, wherein the NTCP mediated activities include bile acid uptake in the liver, and HBV and/or HDV interaction.


SEQUENCE LISTING (11 sequences)

SEQ ID NO: 1 - DNA, 1050 bp, Homo sapiens
  source 1..1050: mol_type = other DNA
atggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctag

SEQ ID NO: 2 - DNA, 866 bp, Homo sapiens
  source 1..866: mol_type = other DNA
cccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaaagtgtctgataagcatcttaaagtcactcaattcctgaactaatcaatatatatgtttaacccattactcaaatacccaaatcccattccaagttttgtgacccaaaagagaaataaatgctcacaagtgctgtagaattaaacttcagaagttctaacctaaaaagttcagatcctattccttcccttttgacattattgggatgatgctcccgaaaagtcaaatttgacatcaagtatgcaaaagtgaacacagtaagatgcaatcaggcaaaacaaactcaaaaaatagctaatgaaatgaaaaaactgggcgaatgcatcatgttagtagaggaggaaaacttttgacaaggaaaaaccaggaaacaaacacatacattaacacaatgttacctcactaataatctcttttttaagttcagtaggtatttggttttaacacaaggcgcccccaaaactgggtacagcaaactactgccaaaatgagtcgtttcccaattcaaagaaaaatgttcaaatacctagaattcacaaatttcaaattgtctctaataaaatttaaacattttgcatatca

SEQ ID NO: 3 - RNA, 19 nt, synthetic construct
  source 1..19: mol_type = other RNA
tccccaacttagaatttgc

SEQ ID NO: 4 - RNA, 19 nt, synthetic construct
  source 1..19: mol_type = other RNA
gcgtcatcctggtgttcat

SEQ ID NO: 5 - RNA, 19 nt, synthetic construct
  source 1..19: mol_type = other RNA
gctttctgctgggttatgt

SEQ ID NO: 6 - RNA, 19 nt, synthetic construct
  source 1..19: mol_type = other RNA
ctttccacctgaagtcatt

SEQ ID NO: 7 - DNA, 19 bp, Homo sapiens
  source 1..19: mol_type = other DNA
tccccaacttagaatttgc

SEQ ID NO: 8 - DNA, 19 bp, Homo sapiens
  source 1..19: mol_type = other DNA
gcgtcatcctggtgttcat

SEQ ID NO: 9 - DNA, 19 bp, Homo sapiens
  source 1..19: mol_type = other DNA
gctttctgctgggttatgt

SEQ ID NO: 10 - DNA, 19 bp, Homo sapiens
  source 1..19: mol_type = other DNA
ctttccacctgaagtcatt

SEQ ID NO: 11 - protein, 349 aa, Homo sapiens
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA